(19) United States
(12) Patent Application Publication
(10) Pub. No.: US 2010/0003344 A1
(43) Pub. Date: Jan. 7, 2010

(54) Cosmetic Skincare Method Employing Mechanical Stresses

(75) Inventor: Guillaume Cassin, Villebon sur Yvette (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/085,225

(22) PCT Filed: Nov. 17, 2006
(86) PCT No.: PCT/EP2006/068630

§ 371 Date: Aug. 15, 2008

Related U.S. Application Data
(60) Provisional application No. 60/741,912, filed on Dec. 5, 2005.

(30) Foreign Application Priority Data
Nov. 21, 2005 (FR) 0553527

(51) Int. Cl. A61K 8/27; A61K 8/64; A61K 8/19; A61Q 19/00

(52) U.S. Cl. 424/630; 514/2; 514/8; 514/12; 514/18; 514/17; 514/16; 514/15; 514/14; 424/642; 424/639

(57) ABSTRACT

The present invention concerns in particular a cosmetic skincare method comprising the simultaneous or sequential application: (i) of a composition comprising at least one agent increasing the expression of mechano-receptors in the cells of the skin; (ii) of a device intended to apply and/or maintain, controlledly, mechanical stresses to/on the skin. It also relates to a cosmetic kit comprising at least a composition comprising at least one agent increasing the expression of mechanoreceptors in the cells of the skin; and a device intended to apply and/or maintain, controlledly, mechanical stresses to/on the skin. The joint use of said composition and said device makes it possible to potentiate and/or prolong the effect of mechanical stresses induced by the application of said device on the improvement in the thickness of the skin, the improvement in the radiance of the complexion, the improvement in the density of the skin and/or the improvement in the mechanical properties of the skin (firmness, elasticity, tonicity) and/or to promote regeneration of the skin and/or its cicatrization.

The field of the invention concerns the enhancement of the appearance of the skin of the face and/or body, and/or the appearance of the complexion.

The present invention concerns in particular a cosmetic skincare method comprising the simultaneous or sequential application:
(i) of a composition comprising at least one agent increasing the expression of mechanoreceptors in the cells of the skin;
(ii) of a device intended to apply and/or maintain, controlledly, mechanical stresses to/on the skin.

It also relates to a cosmetic kit comprising at least
a composition comprising at least one agent increasing the expression of mechanoreceptors in the cells of the skin; and
a device intended to apply and/or maintain, controlledly, mechanical stresses to/on the skin.

The joint use of said composition and said device makes it possible to potentiate and/or prolong the effect of mechanical stresses induced by the application of said device on the improvement in the thickness of the skin, the improvement in the radiance of the complexion, the improvement in the density of the skin and/or the improvement in the mechanical properties of the skin (firmness, elasticity, tonicity) and/or to promote regeneration of the skin and/or its cicatrization.

The concern to preserve for as long as possible a young-looking skin, with good mechanical properties (e.g. firmness, elasticity), and a radiant complexion is a preoccupation of the majority of women and is also, increasingly, affecting men.

The invention is directed in particular to the treatment of the skin of the face and/or neck. However, the compositions of the invention can also be applied to areas of the body exhibiting a loss of elasticity and/or firmness, such as the stomach and the thighs.

The skin constitutes a physical barrier between the body and its environment. It is composed of two tissues: the epidermis and the dermis.

The epidermis is a keratinizing multi-layered epithelium which undergoes continual renewal. Keratinocytes make up the primary epidermal cell population and are responsible for maintaining the epithelial structure and its barrier function. The epidermis rests on an acellular basal membrane, called the dermoepidermal junction, which ensures cohesion with the dermis.

The epidermis is composed of a number of strata of cells, the deepest of which is the basal stratum, which is composed of undifferentiated cells. Over time these cells will undergo differentiation and will migrate towards the surface of the epidermis, thereby making up the different epidermal strata, until, at the surface of the epidermis, they will form the corneocytes, which are dead cells which are removed by desquamation. This surface loss is compensated by the migration of cells from the basal stratum towards the surface of the epidermis. The process is one of continuous renewal of the skin.

The dermis is an elastic and compressible conjunctive support tissue of mesodermal origin and consists primarily of fibroblasts and an extracellular matrix which is composed of fibrous proteins (collagens and elastin) and non-fibrous proteins (proteoglycans and glycoproteins). The dermis is a feeder tissue for the epidermis, but also plays a fundamental part in the development and growth of the epidermis, and also in its differentiation. The fibroblasts and the extracellular matrix also influence the mechanical properties of the skin, particularly its elasticity, tonicity and firmness. The fibroblasts and the extracellular matrix also influence the density of the skin.

The homeostasis of the skin, and in particular of the epidermis, results from a finely regulated balance between the processes of proliferation and differentiation of the cells of the skin. These proliferation and differentiation processes are perfectly regulated: they participate in the renewal and/or regeneration of the skin and lead to the maintenance of a constant skin thickness, and in particular a constant epidermal thickness. This homeostasis of the skin is also involved in maintaining the mechanical properties of the skin.

However, this homeostasis of the skin can be affected by certain physiological factors (age, menopause, hormones, etc.) or environmental factors (UV stress, pollution, oxidizing stress, irritant stress, etc.). The regenerative potential of the epidermis becomes less great: the cells of the basal layer divide less actively, which leads in particular to a slowdown and/or decrease in epidermal renewal. Consequently, cellular renewal no longer compensates for the loss of the cells removed at the surface, leading to atrophy of the epidermis and/or to a decrease in the thickness of the skin and/or a loss of elasticity and/or tonicity and/or firmness of the skin and/or the formation of wrinkles or fine lines.

These clinical signs are visible on the skin of the face and/or neck, but also on the skin of the body, particularly in areas which exhibit a loss of firmness and/or elasticity, such as the stomach and/or thighs.

The alterations in epidermal homeostasis are also manifested in a dull and/or poorly defined appearance to the complexion of the skin.

This phenomenon may be accentuated by the menopause: women complain of their skin tightening and becoming dry, or even of the appearance of xerosis. The hormonal deficits associated with the menopause are accompanied in particular by a drop in metabolic activity, which can result in a decrease in the proliferation of the keratinocytes and in an increase in epidermal differentiation.

The alterations in epidermal homeostasis may also be manifested in less effective regeneration of the skin and/or less effective cicatrization.

It is known that mechanical tensions may influence various functions necessary to the development and homeostasis of the tissues.

The mechanical tensions are transmitted in the cell in the form of biochemical signals via membrane receptors or mechanoreceptors. These include the integrins (Pommerenke et al., Eur J Cell Biol 1996 June; 70(2): 157-64), PECAM1 receptors (Fujiwara et al., Cell struct funct 2001 February; 26(1): 11-17) or else PDGF growth factor receptors (Li et al., Cell Signal 2000 July; 12(7): 435-45).

The tensions, by inducing mechanical perturbation of these receptors, in a first step trigger activation of multiple second messengers. The tensions activate, in particular, protein tyrosine kinase (PTK), protein kinase C (PKC), the G proteins rac and cdc42, or induce the release of calcium flows. The activation of these various signalling pathways leads to the activation of protein kinases from a single family, the MAPkinases, Erk1, Erk2 and p38. The MAPK, once activated, induce the activation of specific transcriptional factors which regulate the expression of numerous genes involved in the homeostasis of keratinocytes. These activation mechanisms are, moreover, well regulated: in the course of tensions, in particular, Erk induces the expression of MAPK phosphatases, which are known to inhibit Erk. This process allows the cells to control the signals induced by the tensions, and to prevent pathological hyperproliferation of the keratinocytes.

This is the context in which the Applicant conceived of using, in combination with a device intended to apply and/or maintain, controlledly, mechanical stresses to/on the skin, a composition comprising at least one agent increasing the expression and therefore the number of mechanoreceptors in the cells of the skin, for the purpose of sensitizing the cells to mechanical stresses induced and/or maintained by the device and thus of making it possible to potentiate and/or increase and/or prolong the effect of these mechanical stresses on the improvement in the homeostatis of the skin, the improvement in the thickness of the skin, the improvement in the radiance of the complexion, the improvement in the density of the skin and/or the improvement in mechanical properties of the skin, in particular its firmness, its elasticity and/or its tonicity, and/or to promote regeneration of the skin and/or its cicatrization. By ‘to sensitize the cells’ is meant to increase their capacity to respond to mechanical stresses.

This combination is also advantageous in that it makes it possible to limit the intensity of the mechanical stresses that are necessary to obtain the desired effect, and to optimize the comfort of the devices used with the aim of applying these stresses to the skin.

By ‘mechanical stresses’ are meant, in particular, tensions and/or tractions and/or pressures on the skin. These mechanical stresses cannot be obtained by superficial brushing of the skin such as is carried out in the course of local, manual application of cosmetic compositions.

The present invention accordingly provides in particular a cosmetic skincare method comprising the simultaneous or sequential application:
(i) of a composition comprising at least one agent increasing the expression of mechanoreceptors in the cells of the skin;
ii) of a device intended to apply and/or maintain, controlledly, mechanical stresses to/on the skin.

By ‘simultaneous application’ is meant, in accordance with the invention, an embodiment in which the composition is contained in a reservoir of said device or else the composition is applied beforehand to said device before the device is applied to the skin. With preference the composition will be contained in said device.

By ‘sequential application’ is meant an embodiment in which the composition and the device are applied successively (immediately) or with a delay to the skin; preferably, the composition will be applied before the device during the first (few) applications, the composition being intended to sensitize the cells to mechanical stresses provided by said device; the order of application is less important during subsequent applications, for a daily treatment involving one to two applications per day.

In particular the invention provides a cosmetic skincare method comprising the simultaneous or sequential application:
(i) of a composition comprising at least one agent increasing the expression of mechanoreceptors in the cells of the skin;
ii) of a device intended to apply and/or maintain, controlledly, a stress selected from a tension, a traction, a pressure and combinations thereof.

### Composition Comprising at Least One Agent Increasing the Expression of Mechanoreceptors in the Cells of the Skin:

By ‘mechanoreceptors’ according to the invention are meant, in particular, membrane receptors which are sensitive to mechanical tensions, in other words membrane receptors which are capable of inducing an intracellular biological response in response to a mechanical perturbation.

They include the integrins (Pommerenke et al., Eur J Cell Biol 1996 June; 70(2): 157-64), PECAM1 receptors (Fujiwara et al., Cell struct funct 2001 February; 26(1): 11-17) or else PDGF growth factor receptors (Li et al., Cell Signal 2000 July; 12(7): 435-45).

Particular interest attaches to the group of the integrins, and especially to the class of β1 integrins which are involved in the sensitivity of the cells to mechanical stresses.

Integrins are adhesion molecules which are involved in cell-cell and cell-matrix interactions. They are heterodimeric receptors composed of two subunits, α and β, which are associated non-covalently. More than 17 chains of subunit α and 8 chains of subunit β have been described, which associate to form 23 different heterodimers.

The transmembrane region of the α subunits is composed of an α helix, very highly conserved from one subunit to another, which is responsible for the function of anchoring the integrin to the membrane, and which participates in signal transduction.

The cytoplasmic region of the β subunits, which is very highly conserved from one subunit to another, is responsible on the one hand for the formation of the heterodimer and on the other hand for bonding with the structural proteins of the cytoskeleton; this combination also regulates signal transduction.

The heterodimers of integrins can be classed according to their substrate; it is known in particular that:
heterodimers α1β1 and α2β1 bind to collagen;
heterodimers α4β1, α5β1, α8β1 and αvβ1 bind to fibronectin;
heterodimers α1β1, α2β1, α3β1 and α6β1 bind to laminins.

Collagen, fibronectin and laminins are matrix proteins or proteins of the extracellular matrix which participate in the adhesion of these cells and which play an important part in migration and in cell signalling. In the course of the processes of adhesion and of cell migration, the cells interact with the matrix molecules via membrane receptors and in particular the integrins as described above. This interaction initiates intracellular responses which are involved in cell signalling, cell differentiation, migration and/or cell proliferation.

Peptides mimicking the structure of certain regions of these matrix proteins have been defined in the prior art: described in particular is the use of peptides of fibronectin (WO 03/008438), collagen peptides (WO 03/007905) and peptides of fibronectin (WO 03/077936) in compositions in order to increase cellular adhesion.

By ‘agent increasing the expression of mechanoreceptors in the cells of the skin’ is meant in particular, according to the invention, any agent capable of inducing or of stimulating the expression of mechanoreceptors in the cells of the skin, particularly in the cells of the epidermis and the dermis (e.g. keratinocytes, fibroblasts).

Interest attaches preferably to agents which increase the expression of integrins, and particularly to agents which increase the expression of β1 integrins.

Such agents may be selected according to conventional methods of detection by immunofluorescence or by quantitative RT-PCR. Preference will be given to using the quantitative RT-PCR technique.

The principle of detection by immunofluorescence consists in contacting cells in culture with the agents under test and then in visualizing the effect of said agents on the expression of mechanoreceptors and in particular of integrins (e.g. β1 integrins) by using anti-integrin antibodies and secondary antibodies coupled to a fluorescent marker (fluorescein).

The general principle of the quantitative RT-PCR technique, which is preferred according to the invention, comprises, for example, the following steps:
the concentration of the agents under test are selected on the basis of a cytotoxicity study under the conditions of the assay;
human keratinocytes or fibroblasts are cultivated in a culture medium adapted to these different cell types;
the culture medium is exchanged for the same medium containing or not containing (control) the agent under test at the various concentrations selected;
after 24 h of incubation, for example, the mRNAs are extracted and the traces of DNA are removed by treatment with DNAse, which is subsequently deactivated;
then a reverse-transcription reaction is carried out, followed by quantification, by fluorescence, of the cDNA synthesized;
a first series of Q-PCRs is carried out on the β actin marker (check) in order to verify the homogeneity of the preparations to be compared;
subsequently Q-PCRs are carried out in triplicate using pairs of primers specific for the β-actin sequences, and markers specific for mechanoreceptors and in particular for integrins (e.g. β1 integrins);
next, the differential expression of the integrins is evaluated by fluorescence analysis in amplified DNA;
a selection is made of the agents for which an increase in the fluorescence intensity is obtained, corresponding to an increase in the expression of integrins relative to the control condition (that not treated with the agent).

The PCR reactions (polymerase chain reaction) can be performed in particular by quantitative PCR with the “Light Cycler” system (Roche Molecular Systems Inc.) and in accordance with the procedures recommended by the supplier.

Preferably the agent increasing the expression of mechanoreceptors in the cells of the skin that is present in the composition is an agent increasing the expression of integrins in the cells of the skin.

According to one first embodiment the agent increasing the expression of mechanoreceptors in the cells of the skin is a peptide.

This peptide may increase the expression of mechanoreceptors in the cells of the skin in particular by either (i) binding to the target mechanoreceptors or (ii) by binding to any other membrane receptor capable of inducing an intracellular response leading to an increase in the expression of the target mechanoreceptors, in particular of integrins.

Examples of peptides which are useful according to the invention include, in particular, mimetic peptides of matrix proteins, their homologues or derivatives, which are capable of binding to the target mechanoreceptors.

In particular the mimetic peptides of matrix proteins will be selectable from mimetic peptides of collagen, fibronectin or laminin.

By ‘mimetic peptides of matrix proteins’ are meant peptide sequences which are contained within the native sequences of matrix proteins that are identified as being the key sequences for the function of cellular adhesion, or peptide sequences which are homologous with sequences contained in the native sequences of matrix proteins that are identified as being the key sequences for ensuring the function of cellular adhesion. These key sequences of the matrix proteins are involved in particular in the binding of the proteins with the mechanoreceptors.

These peptides are capable of mimicking the structure of certain regions of said matrix proteins and thus of inducing the same types of signals as them: in particular, they are capable especially of binding to the target mechanoreceptors and of inducing an increase in the expression of said mechanoreceptors.

These peptides will generally have a sequence ranging from 2 to 25 amino acids, in particular from 4 to 16 amino acids.

The peptides useful according to the invention include in particular:

1) fibronectin peptides, their homologues and derivatives, such as the peptides of sequence (AA)ₙ-Leu-Asg-Ala-Pro-(AA)ₙ in which AA is any amino acids or derivative thereof and n is between 0 and 2, and in which the amino acids may be in the L (levogyratory), D (dextrogyratory) or DL form. Peptides of this kind are described in patent application WO 03/008438, which is incorporated by reference in the present invention.

Preference will be given to using the hexapeptide of sequence Lys-Leu-Asp-Ala-Pro-Thr, which is homologous with a sequence contained in the subunit III of fibronectin, a homologue or a derivative of this peptide. This peptide is sold by Vincience under the name VINCI 02®. It stimulates the expression of β1 integrins on cells in culture, as described in patent application WO 03/008438.

2) collagen peptides, their homologues and derivatives, such as the peptides of sequence (Gly-Pro-Gln)ₙ-NH₂ in which n is between 1 and 3, and in which the amino acids may be in the L, D or DL form, as described in patent application WO 03/007905, which is incorporated in the present application by reference.

Preference will be given to using the hexapeptide of sequence Gly-Pro-Gln-Gly-Pro-Gln whose sequence is contained in the sequence of collagen, or a homologue or derivative of this peptide. This peptide is sold by Vincience under the name COLLAXYL®.

It stimulates the expression of β1 integrins when it is applied to the skin ex vivo as described in Perrin et al. (Int J Tissue React, 2004; 26(3-4): 97-104).

3) laminin peptides, their homologues and derivatives, such as:
the hexapeptide Serilesine® sold by Lipotec, whose sequence is homologous with a sequence contained in the α chain of laminin. This peptide is described as increasing the expression of α6 integrins in fibroblasts and keratinocytes in culture;
peptides of sequence X₁-Y-Phe-Thr-X₂-Ala-Thr-Z-Ile-X₃-Leu-X₄-Phe-Leu-X₅ in which X₁, X₂, X₃, X₄ and X₅=Arg, Lys or His, Y=Asp or Glu, and Z=Asn or Gln, as described in patent application WO 03/077936, incorporated by reference in the present application. In particular, the oligopeptide Arg-Asp-Phe-Thr-Lys-Ala-Thr-Asn-Ile-Arg-Leu-Arg-Phe-Leu-Arg, whose amino acid sequence is homologous with a sequence contained in the sequence of laminin. This peptide stimulates the expression of β1 integrins, as described in patent application WO 03/077936. This peptide is sold by Vincience under the name VINCI 01®.

The invention accordingly provides in particular a cosmetic skincare method comprising the simultaneous or sequential application:
(i) of a composition comprising at least one agent increasing the expression of mechanoreceptors in the cells of the skin;
(ii) of a device intended to apply, controlledly, mechanical stresses to the skin;
   in which the agent increasing the expression of mechanoreceptors in the cells of the skin is a peptide selected from:
a) a fibronectin mimetic peptide of sequence (AA)ₙ-Leu-Asg-Ala-Pro-(AA)ₙ in which AA is any amino acid or derivative thereof and n is between 0 and 2;
b) a collagen mimetic peptide of sequence (Gly-Pro-Gln)ₙ-NH₂ in which n is between 1 and 3;
c) a laminin mimetic peptide of sequence X₁-Y-Phe-Thr-X₂-Ala-Thr-Z-Ile-X₃-Leu-X₄-Phe-Leu-X₅ in which X₁, X₂, X₃, X₄ and X₅=Arg, Lys or His, Y=Asp or Glu and Z=Asn or Gln;
   and derivatives thereof.

In particular the peptide is selected from:
d) a fibronectin mimetic hexapeptide of sequence Lys-Leu-Asp-Ala-Pro-Thr;
e) a collagen mimetic hexapeptide of sequence Gly-Pro-Gln-Gly-Pro-Gln;
f) a laminin mimetic oligopeptide of sequence Arg-Asp-Phe-Thr-Lys-Ala-Thr-Asn-Ile-Arg-Leu-Arg-Phe-Leu-Arg; homologues thereof and derivatives thereof.

These peptides may be of natural or synthetic origin.

By ‘natural origin’ is meant a peptide in the pure state or in solution at different concentrations which is obtained by various processes of extraction from a keratinic material (skin, nail, hair, especially hair) of natural origin or from conjunctive tissues.

By ‘synthetic origin’ is meant a peptide in the pure state or in solution at different concentrations which is obtained chemically or by production in an organism following introduction into said organism of the elements necessary for said production.

These peptides may be obtained by chemical or enzymatic synthesis from the constituent amino acids or their derivatives, or by managed hydrolysis of natural (vegetable or animal) proteins or else by biotechnology in accordance with conventional techniques.

By ‘homologue of these peptides’ is meant, in particular, any peptide sequence identical to at least 50%, preferably to at least 80% and more preferably to at least 95% of said peptide sequences identified above, in the same species or in a different species; in the latter case, it is also denoted by ‘orthologous polypeptide’.

Also, ‘percentage identity’ between two peptide sequences or amino acid sequences is intended to denote a percentage of amino acid residues which are identical between the two sequences under comparison that is obtained after the best alignment, i.e. the optimum alignment achieved, for example, using the Smith-Waterman local homology algorithm (1981, Ad. App. Math. 2: 482), using the Neddleman-Wunsch local homology algorithm (1970, J. Mol. Biol. 48: 443), using the Pearson-Lipman similarity search method (1988, Proc. Natl. Acad. Sci. USA 85: 2444), or using computer software that utilizes these algorithms (GAP, BESTFIT, BLAST P, BLAST N available on the site NCBI, FASTA and TFASTA in the Wisconsin Genetics Software Package, Genetics Computer Group, 575 Science Dr, Madison, Wis.).

By ‘derivative’ of these peptides is meant, in particular, a peptide modified by acylation on its N-terminal function and/or by esterification on its C-terminal function.

The peptides may be solubilized beforehand in one or more cosmetically acceptable solvents such as water, propylene glycol, butylene glycol, ethoxylated or propoxylated diglycols, ethanol, propanol or isopropanol.

They may alternatively be solubilized in a cosmetic vector such as liposomes, or adsorbed on organic or inorganic supports.

These peptides are used in the composition according to the invention in an amount effective for obtaining the required effect, particularly for increasing the expression of integrins.

The amount of peptides which can be used according to the invention may range from 0.01% to 20% by weight relative to the total weight of the composition, preferably from 0.1% to 10% by weight relative to the total weight of the composition.

According to another embodiment, the agent increasing the expression of mechanoreceptors and in particular of integrins that is present in the composition employed in the method of the invention is selected from zinc salts, manganese salts, copper salts, derivatives thereof and mixtures thereof.

By ‘salts’ are meant organic or inorganic salts.

Possible organic salts include gluconate, carbonate, acetate, citrate, oleate or oxalate.

Possible inorganic salts include mineral salts such as chloride, borate, nitrate, phosphate or sulphate.

Alternatively it will be possible to use zinc, copper and manganese in an ionic form, in the form of salts or in the form of natural extracts, plant extracts or extracts from microorganisms, particularly bacterial extracts, which are rich in zinc, copper and manganese.

Use will be made in particular of organic salts of zinc, of copper or of manganese, derivatives thereof or mixtures thereof.

Preference will be given to using at least one gluconate salt selected from a zinc gluconate, a copper gluconate, derivatives thereof and mixtures thereof.

These zinc, copper and manganese gluconates have been described as being capable of increasing the expression of integrins, in particular the α2, α3, α6, αv and β1 integrins, on keratinocytes in culture or in reconstructed skins (Tenaud et al., British Journal of Dermatology, 1999: 140; 26-34).

These gluconate salts are sold in particular by Labcatal.

The zinc salts, copper salts or manganese salts are present in the composition in an amount effective for obtaining the required effect, particularly for increasing the expression of integrins. It will be possible to use an amount ranging from 0.01% to 20% by weight relative to the total weight of the composition, preferably from 0.1% to 10% by weight relative to the total weight of the composition.

By ‘derivatives’ are meant salts which are complexed with sugars or with amino acids.

The composition according to the invention comprises a physiologically acceptable medium, in other words a medium which is compatible with the skin of the face and/or body. It is preferably a cosmetically acceptable medium, in other words a medium which has a colour, odour and feel that are pleasant and which does not give rise to any unacceptable discomfort (stinging, tautness, redness) that might dissuade the consumer from using this composition.

The composition according to the invention may be a bodycare or facecare composition or a makeup composition.

The composition according to the invention may be in any of the formulated forms conventionally used for topical application, and particularly in the form of dispersions of the aqueous gel or lotion type, emulsions with a liquid or semi-liquid consistency of the milk type, obtained by dispersing a fatty phase in an aqueous phase (O/W) or conversely (W/O), or of suspensions or emulsions with a soft, semi-solid or solid consistency, of cream or gel type, or in the form of a serum or stick, or else of multiple emulsions (W/O/W or O/W/O), of microemulsions, of ionic and/or nonionic vesicular dispersions, or of wax/aqueous phase dispersions. These compositions are prepared in accordance with the customary methods.

Oils which can be used in the composition according to the invention include the following:
hydrocarbon oils of animal origin, such as perhydrosqualene;
hydrocarbon oils of vegetable origin, such as liquid triglycerides of fatty acids containing 4 to 10 carbon atoms, or else, for example, vegetable oils such as apricot kernel oil and shea butter oil;
synthetic esters and ethers, especially those of fatty acids, such as the oils of formulae R¹COOR² and R¹OR² in which R¹ represents the residue of a fatty acid containing 8 to 29 carbon atoms and R² represents a branched or unbranched hydrocarbon chain containing 3 to 30 carbon atoms;
linear or branched hydrocarbons of mineral or synthetic origin, such as volatile or non-volatile liquid paraffins and derivatives thereof, isohexadecane, isododecane, petroleum jelly, polydecenes, hydrogenated polyisobutene such as Parléam® oil;
natural or synthetic essential oils;
branched fatty alcohols having 8 to 26 carbon atoms, such as octyldodecanol;
partially hydrocarbon-modified and/or silicone-modified fluoro oils like those described in document JP-A-2-295912;
silicone oils such as volatile or non-volatile polydimethylsiloxanes (PDMS) having a linear or cyclic silicone chain which are liquid or pasty at ambient temperature, especially cyclopolydimethylsiloxanes (cyclomethicones) such as cyclohexasiloxane and cyclopentasiloxane; polydimethylsiloxanes containing alkyl, alkoxy or phenyl groups pendantly or at the end of a silicone chain, these groups having 2 to 24 carbon atoms; phenyl silicones such as phenyltrimethicones, phenyldimethicones, phenyltrimethylsiloxydiphenylsiloxanes, diphenyldimethicones, diphenylmethyldiphenyltrisiloxanes, 2-phenylethyl trimethylsiloxysilicates, and polymethylphenylsiloxanes; and
mixtures thereof.

The other fatty substances which may be present in the oily phase are, for example, fatty acids containing 8 to 30 carbon atoms, such as stearic acid, lauric acid, palmitic acid and oleic acid; linear fatty alcohols such as cetyl alcohol and/or stearyl alcohol; pasty fatty substances such as lanolin; waxes; and gums such as silicone gums (dimethiconol).

These fatty substances may be selected variously by the skilled worker in order to prepare a composition having the desired properties of, for example, consistency or texture.

This composition may further comprise various adjuvants commonly used in the field of cosmetology, such as emulsifiers, including esters of fatty acids and of polyethylene glycol, esters of fatty acids and of sorbitan which are optionally polyoxyethylenated, polyoxyethylenated fatty alcohols and the esters or ethers of fatty acid and of sugars such as sucrose or glucose; fillers; preservatives; sequestrants; fragrances; and thickeners and/or gelling agents, such as homopolymers and copolymers of acrylic acid, homopolymers and copolymers of acrylamide and/or of 2-acrylamido-2-methylpropanesulphonic acid (AMPS), modified AMPS products (Aristoflex LNC and SNC), and xanthan gum.

Fillers include, for example, particles of polyamide (Nylon) in spherical or microfibre form; microspheres of polymethyl methacrylate; ethylene-acrylate copolymer powders; expanded powders such as hollow microspheres and, in particular, the microspheres formed from a terpolymer of vinylidene chloride, acrylonitrile and methacrylate which are sold under the name Expancel; powders of natural organic materials such as powders of starch, particularly of maize starch, wheat starch or rice starch, crosslinked or non-crosslinked, such as powders of starch crosslinked with octenylsuccinic anhydride; silicone resin microbeads such as those sold under the name Tospearl by Toshiba Silicone; silica; metal oxides such as titanium dioxide or zinc oxide; mica; hollow hemispherical particles of silicone such as NLK506 sold by Takemoto Oil and Fat; and mixtures thereof.

The skilled worker will of course take care to select this or these optional additional compounds and/or their amount in such a way that the advantageous properties of the composition according to the invention are not, or not substantially, adversely affected by the intended addition.

The composition according to the invention is applied in accordance with the typical techniques, for example by application of creams, gels, serums or lotions to the skin it is intended to treat, in particular the skin of the body, face and/or neck.

The composition according to the invention may also contain actives having a complementary effect to the combination according to the invention, such as at least one compound selected from desquamating agents, moisturizing agents, agents which stimulate the proliferation and/or differentiation of keratinocytes, agents which stimulate the synthesis of collagen and/or elastin or prevent their breakdown, depigmenting agents, anti-glycation agents, agents which stimulate the synthesis of glycosaminoglycans, antioxidants and free-radical scavengers, and mixtures thereof.

Examples of such actives are as follows: retinol and its derivatives such as retinyl palmitate; ascorbic acid and its derivatives such as magnesium ascorbyl phosphate and ascorbyl glucoside; tocopherol and its derivatives such as tocopheryl acetate; nicotinic acid and its precursors such as nicotinamide; ubiquinone; glutathione and its precursors such as L-2-oxothiazolidine-4-carboxylic acid; plant extracts and especially extracts of rock sapphire and of olive leaf; algal extracts and especially laminaria extracts; bacterial extracts; sapogenins such as diosgenin and dioscorea extracts, in particular wild yam extracts, which contain them; α-hydroxy acids; β-hydroxy acids, such as salicylic acid and n-octanoyl-5-salicylic acid; oligopeptides and pseudodipeptides and their acyl derivatives, especially {2-[acetyl(3-trifluoromethylphenyl)amino]-3-methylbutyrylaminoacetic}acid and the lipopeptides sold by Sederma under the trade names Matrixyl 500 and Matrixyl 3000; lycopene; and mixtures thereof.

Device Intended to Apply and/or Maintain, Controlledly, Mechanical Stresses to/on the Skin

By ‘application and/or maintenance, controlledly, of mechanical stresses’ is meant a stimulation of the skin by means of a device, it being possible for said device to be applied to skin which is not subject to a mechanical stress (referred to as application of a stress), or to skin which is already subject to a mechanical stress of tension or traction type in particular (referred to in this case as maintenance of the stress).

These mechanical stresses may be the direct or indirect result of applying and/or maintaining to/on the skin a mechanical, electrical or ultrasonic stimulation or combination thereof by means of at least one device. By ‘indirect result’ is meant, for example, a mechanical stress induced by stimulation of the subcutaneous muscles, electrically for example (e.g. electrostimulation), by means of a device.

By ‘mechanical stresses’ are meant in particular in accordance with the invention a stress selected from a tension, a traction, a pressure, and combinations thereof.

In particular the device according to the invention intended to apply and/or maintain mechanical stresses to/on the skin is selected from a device inducing mechanical stimulation, electrical stimulation, ultrasonic stimulation, and combinations thereof.

Certain devices will be favoured in dependence on the mechanical stress that they induce and on the target application.

By way of example, preference will be given to using a device which generates tractions for the purpose of inducing/stimulating cellular renewal and thus of combating ageing-related alterations.

And it will be possible with preference to use devices that generate pressures in order to reduce the accumulation of extracellular matrix and thus to promote regeneration of the skin and/or its cicatrization, in particular by combating the development of hypertrophic scars or keloids.

The stimulation of the skin in accordance with the invention by means of a device for applying and/or maintaining the mechanical stresses may be the result of a mechanical, electrical or ultrasonic action or combination thereof.

Preference will be given to using a stimulation induced by a mechanical device.

### Stimulation Induced by a Mechanical Device

According to one first embodiment the device inducing mechanical stimulation is a massage instrument or pressure applicator.

### a) Massage Instruments or Pressure Applicators

These devices may be manual, which is to say that they have no need, in order to operate, for provision of external energy other than a simple application of the device to the skin, optionally with a back-and-forth movement (massage), or may be provided with an electric motor operating either on the mains supply or on a battery supply.

According to a first embodiment the device may be a massage instrument selected from a manual massage instrument or a massage instrument requiring provision of external energy.

Examples that may be mentioned of manual instruments include the apparatus developed or sold by the following companies: Environ (Environ Cosmetic Roll-Cit®); Repechage (Repechage Facial Lift On The Go®); Leaf & Rusher (Leaf & Rusher DermaRoller®).

These instruments may take the form of massage balls or rollers optionally containing pins on their surface.

Examples that may be mentioned of instruments requiring provision of external energy include the apparatus developed or sold by the following companies: WinHealth (Magic Youth Wand Eye Roller®); HoMedics (FAC-100 Facial Spa®); LPG (Lift6®).

These massage instruments may be assisted mechanically, in other words comprising manual control means, comprising a treatment head connected to a suction circuit, said treatment head comprising two mutually opposed surfaces, and said suction circuit allowing the formation, as a result of the reduced pressure created, of a fold of skin which will then be displaced on the surface of the human body.

Apparatus such as the Lift6® make it possible to carry out specific massages, which are obtained by the use of massage heads connected to a suction circuit. The massage heads comprise a box, a chamber and two transverse surfaces composed of rollers driven positively in rotation; apparatus of this kind is described for example in French Patent Applications FR2579100, FR2589726, FR2612395, FR2723310, FR2752159, FR2768051 and FR2809952.

An example that may be mentioned of a mechanical stimulation device generating a pressure is an apparatus comprising an applicator that is configured to apply a pressure pulse to the surface of the skin, having at least one phase of negative pressure relative to the ambient pressure, such as the apparatus described in Patent Application WO0697925.

This phase of negative pressure relative to the ambient pressure may have a duration of 0.1 to 100 milliseconds and an intensity of 0.1 bar to 10 bars below the ambient pressure.

### b) Supports, Especially Adhesive Supports, Maintaining Tensions

According to another embodiment, the device intended to generate and/or maintain mechanical stresses on the skin, in particular to maintain tensions already applied to the skin, is a support, in particular an adhesive support, capable of maintaining a tension on the skin.

This support will be applied to a stretched or drawn area of skin so as to maintain the skin under tension.

This support will be characterized in particular by an elastic modulus greater than or equal to 500 MPa, in particular ranging from 500 to 10 000 MPa, preferably from 500 to 2000 MPa, more preferably still from 500 to 1500 MPa. This modulus may in particular be determined by dynamic mechanical analysis using, for example, the DMA 2980 (Dynamic Mechanical Analyzer) sold by TA Instruments.

This support may for example be a synthetic support.

As an example of a support, in particular an adhesive support, mention may be made of patches.

Patches generally have a composite structure in the form of layers.

Advantageously they contain a reservoir comprising the composition or active which is intended to be released on the skin at the time the patch is applied.

The following may be mentioned by way of example:
controlled-release patches with a hydrophobic polymeric matrix (FR 2 738 744 L'Oréal);
reservoir-type patches containing a reservoir of active substance, a diffusion membrane and an adhesive layer;
optimized-adhesion patches, comprising a layer of hydrophobic polymer attached to a support layer and containing particles of active compound, particles of oil and particles of a water absorber (FR 2 761 889 L'Oréal);
   and advantageously the ‘reservoir’ systems which allow controlled release of said active substance.

The patch generally comprises at least one polymeric matrix with a surface which is adhesive or apt to become so, particularly after wetting, and is intended to be placed in contact with the skin.

By ‘polymeric matrix’ is meant a layer of hydrophobic or hydrophilic polymer which may be composed of a matrix which is self-adhesive (to dry skin and/or to wetted skin).

The ‘hydrophobic’ polymeric matrices making up ‘conventional’ patches are based in particular on polyacrylic or polyvinyl adhesive, on a silicone, polyurethane, styrene or isoprene polymer, whose crosslinking is preferably partial, so as to provide it with adhesion without the need for an additional adhesive layer. It is also possible to use an adhesive matrix made of latex, butyl or any other elastomeric adhesive.

The surface of the matrix that is intended to come into contact with the skin may be smooth or may have roughness or reliefs.

Preferably the polymeric matrix is deposited on a support.

The support may be an ‘occlusive’ support. By way of example the support is composed of a thermoplastic material, selected from high-density and low-density polyethylenes, polypropylenes, polyvinyl chlorides, ethylene-vinyl acetate copolymers, polyesters and polyurethanes, or of a complex of such materials. These materials may also be present in layered form with at least one metal foil such as an aluminium foil.

The support layer may be of any appropriate thickness that will provide the desired support and protection functions. Preferably the thickness of the support layer is between approximately 20 μm and approximately 1.5 mm. With advantage the support layer is sufficiently flexible to be able to conform perfectly to the profile of the skin without causing any sensation of discomfort to the user.

With preference, however, the support is ‘non-occlusive’. In this latter case, use is made advantageously of a support composed of paper, of a porous or perforated thermoplastic material, of a woven fabric, of a non-woven fabric or of a perforated non-woven fabric.

Preferably the patch comprises at least one protective sheet which can be peeled off after application of the patch.

The patch may be packaged in a carton or in a protective envelope formed of two sheets of an impervious paper/plastic film complex, the paper being coated with a cold-seal adhesive and the sheets being sealed around the patch by contact of the adhesive-coated faces.

### Stimulation Induced by an Electrical Device

Devices which generate electrical stimulations, or electrostimulations, are conventionally used to act on the muscle tone, by contraction of the muscles. This muscular contraction may have the effect of generating mechanical stresses locally.

Electrostimulation uses a discontinuous low-voltage current which is transmitted by electrodes connected to an apparatus.

Electrostimulators generally comprise electrodes with a surface applied to the skin in the areas to be treated, said electrodes being connected to an electrical generator which applies electrical stimulations with variable frequencies ranging from 0.1 to 100 Hz, in particular from 0.2 to 60 Hz, and preferentially from 0.2 to 20 Hz.

An example that may be mentioned for stimulation in the form of electrical stimulation is a micro dermal tone skin stimulator system such as that described in Patent Application WO 06116728. The output pulses are adjustable within a range of from approximately 0.3 to 8 Hertz.

### Stimulation Induced by an Ultrasound or Sonophoresis Device

Ultrasound frequencies vary from 20 kHz to 10 MHz.

Use will be made generally of low-frequency (e.g. 20 kHz -300 kHz) or medium-frequency (e.g. 300 kHz-3 MHz) devices which are known to have an effect of muscle massage, stimulation of the microcirculation, and cell contraction.

An example that may be mentioned of a device for stimulation in the form of ultrasound is a stimulator which applies to the skin a combined stimulus of ultrasound and low frequencies, such as that described in Patent Application WO 06101295.

Mention may also be made for example of the Sonic Peeler®, which is sold by Neps Inc. and, when applied to the skin, generates ultrasonic vibrations.

Mention may additionally be made of the device described in Patent Application US20050191252, which combines an ultrasound stimulation of the order of 5-6 MHz with an electrical stimulation of iontophoresis type.

According to one particular embodiment of the invention it will also be possible to use, via at least one device, a combination of these different types of stimulation in order to improve and/or optimize the application and/or maintenance of mechanical stresses to/on the skin.

With preference a single device will induce a number of types of stimulation.

It will thus be possible to use a device generating:
a mechanical stimulation and an electrical stimulation (e.g. electrostimulating patches);
an electrical stimulation and an ultrasonic stimulation;
a mechanical stimulation and an ultrasonic stimulation.

According to one particular embodiment of the invention the composition comprising at least one agent increasing the expression of mechanoreceptors in the cells of the skin is contained in a reservoir of said device and is released at the time said device is applied to the skin.

The method according to the invention is intended in particular to promote the homeostasis of the skin and/or improve the mechanical properties of the skin (e.g. firmness, elasticity, tonicity) and/or to promote a radiant complexion and/or to improve the density of the skin and/or regeneration of the skin.

According to one particular embodiment the composition and the device according to the invention will be applicable to people who exhibit a dull and/or poorly defined complexion, in order to promote a radiant complexion.

In another embodiment the composition and the device according to the invention will be applicable to people who exhibit a soft and/or flaccid skin or who have regions of the body exhibiting a loss of elasticity and/or firmness.

In particular it will be possible to apply the composition to the face, stomach and thighs.

The invention likewise provides a method as described above for combating skin ageing, characterized in that a device is used which is intended to apply and/or maintain a traction and/or a tension to/on the skin.

The invention also relates to a method as described above for combating skin ageing, characterized in that a device is used which is intended to apply and/or maintain a pressure to/on the skin.

Advantageously, and in order to obtain a remanent effect over time on the homeostasis of the skin, it will be possible to apply the composition and the device according to the invention twice-weekly, preferably daily, in the morning and/or the evening.

It will be possible for the method according to the invention to consist, for example, in a daily application, in the morning and/or evening, simultaneously or sequentially (successively or with a delay), of the composition and of the device.

The application is made to the areas of the face and/or body to be treated.

The device intended to generate mechanical stresses is applied to the skin for a period preferably greater than or equal to 1 minute and possibly, for example, from several minutes (particularly in the case of massage with a massage instrument) to 1 or more hours (in the case for example of a patch providing controlled release of the active).

The invention likewise provides a cosmetic kit comprising at least:
a composition comprising at least one agent increasing the expression of mechanoreceptors in the cells of the skin; and
a device intended to apply and/or maintain, controlledly, mechanical stresses to/on the skin.

In particular the device intended to apply and/or maintain, controlledly, mechanical stresses to/on the skin is selected from a device inducing mechanical stimulation, electrical stimulation, ultrasonic stimulation and combinations thereof.

In particular the device intended to apply and/or maintain, controlledly, mechanical stresses to/on the skin in this kit is a massage instrument selected from a manual massage instrument or a massage instrument requiring provision of external energy.

According to one alternative the device intended to maintain tensions on the skin in this kit is a support, in particular an adhesive support, capable of maintaining a tension on the skin.

A preferred support which can be used in the kit according to the invention is a patch.

According to one particular kit of the invention the composition is contained in a reservoir of said device intended to apply and/or maintain, controlledly, mechanical tensions to/on the skin.

Examples of agents increasing the expression of mechanoreceptors and present in the composition have been described above.

Use will be made in particular, in the composition forming part of the care kit according to the invention, of an agent selected from a peptide increasing the expression of mechanoreceptors in the cells of the skin, as described above, or zinc salts, copper salts, manganese salts, derivatives thereof and mixtures thereof.

Examples of these compounds have been described above.

The invention likewise provides for the joint use of a composition comprising at least one agent increasing the expression of mechanoreceptors in the cells of the skin and of a device intended to apply and/or maintain, controlledly, mechanical stresses to/on the skin, said composition being intended to sensitize the cells of the skin to mechanical stresses induced by the application of said device.

This joint use has the effect in particular of potentializing and/or prolonging the effect of mechanical stresses induced by the application of said device on the improvement in the thickness of the skin, the improvement in the radiance of the complexion, the improvement in the density and/or the improvement in the mechanical properties of the skin, (firmness, elasticity, tonicity) and/or the improvement in the regeneration of the skin and/or its cicatrization.

The invention will now be described with reference to the following examples, which are given by way of illustration and not of limitation. In these examples, unless indicated to the contrary, the amounts are expressed as weight percentages.

### EXAMPLES

### Example 1

### Effect of the Combination of an Agent Increasing the Expression of Integrins and a Device Intended to Apply and/or Maintain a Tension to/on the Skin

a) Device Intended to Maintain a Tension

The effect of the combination between an active increasing the expression of integrins (e.g.: manganese gluconate or hexapeptide Lys-Leu-Asp-Ala-Pro-Thr) and a device maintaining a tension (e.g. patch) on the differential expression of genes involved in the homeostasis of the skin (e.g.: TGFb, keratin 19 and HSP90A) was evaluated by RT-Q-PCR on Episkin® reconstructed epidermides, in comparison to the effect of the peptide and of the patch per se.

A patch is used which has an elastic modulus of greater than 500 MPa. Patches with elastic moduli of 500 MPa, 1000 MPa, 1500 MPa and 2000 MPa are tested.

The Episkin® reconstructed epidermides used are obtained at d 15. They are placed in a maintenance medium for 8 hours. They are subsequently transferred to a DMEM/Ham F12 medium devoid of EGF, of pituitary extract and of foetal calf serum. The epidermides are left in this medium for 24 hours to equilibrate.

The Episkin®s are then pretreated for 4 h and 24 h with 2 μg/ml manganese gluconate or 15 pg/ml hexapeptide Vinci02 (Lys-Leu-Asp-Ala-Pro-Thr) (Vincience), which are known to increase the number of mechanoreceptors (beta1 integrins) on the surface of the keratinocytes. After this pretreatment, a mechanical device of the patch type is applied to the Episkin®s, which are already subject to a tension, in this culture medium and is left in contact with the epidermides for 24 hours in a chamber thermostated at. 37° C. and 40% relative humidity.

At the end of this period the epidermides are withdrawn and extracted for the RT-Q-PCR studies.

Other Episkin®s are treated under the same conditions but, respectively, with the patch alone or with manganese gluconate/hexapeptide alone.

The effect of the products to be tested on the expression of the markers selected was evaluated by RT-Q-PCR, which was carried out on the basis of total RNAs extracted from the epidermides, in accordance with the following protocol:

The first step consists in carrying out a reverse transcription reaction.

This step requires prior treatment of the total RNAs in order to remove traces of potentially contaminating DNA by treatment with the system DNA-free (Ambion). The reverse transcription of the mRNAs to cDNAs takes place in the presence of the oligo(dT) primer and of the Superscript II enzyme (Gibco).

The PCRs (polymerase chain reactions) were carried out by quantitative PCR using the “Light Cycler” system (Roche Molecular Systems Inc.) and in accordance with the recommendations of the supplier. This analytical system allows rapid and high-performance PCR reactions to be carried out providing prior development of the conditions for analysis of the different primers. It is formed of two main components:
a thermocycler: optimized by virtue of the use of glass capillaries and of extremely rapid thermal transfers.
a fluorimeter: this allows the fluorescence intensity incorporated in the DNA to be measured continuously (detection at 521 nm).

The reaction mixture (10 μl final) introduced into the capillaries for each sample is as follows:
2.5 μl of cDNA diluted 1/10
primers of the different markers used
reaction mixture (Roche) containing the taq DNA polymerase enzyme, the SYBR marker and Green I (fluorophore which is intercalated into the double-stranded DNA in the course of the elongation step) and MgCl₂.

The incorporation of fluorescence into the amplified DNA is measured continuously in the course of the PCR cycles. This system enables curves to be obtained of measurement of the fluorescence as a function of the PCR cycles, and thus makes it possible to evaluate a relative expression value for each marker. The number of cycles is determined on the basis of the “exit” points of the fluorescence curves. For a given marker under analysis, the later the exit of a sample (high cycle number), the weaker the initial number of copies of the mRNA. The RE (relative expression) value is expressed in arbitrary units in accordance with the following formula: (½ number of cycles)×10⁶.

### b) Device Intended to Apply a Tension and a Pressure to the Skin

According to one alternative embodiment the patch used in a) is replaced by a TA-XT21 texture analyser device sold by Stable Micro System. This analyser is adapted for application to Episkin® and has a hemispherical head.

In this way it is possible to apply to Episkin® a tension and a compression (pressure) at different conditions ranging from 5 MPa to 90 MPa, in particular MPa, 10 MPa, 20 MPa, 50 MPa and 90 MPa.

The operational protocol followed is the same as that described in a).

### Example 2

### Compositions and Application Routine

Oil-in-water emulsion
Phase A

| | |
|---|---|
| Glyceryl stearate (and) PEG-100 stearate | 2.00 g |
| (Arlacel 165FL): | |
| Dimyristyl tartrate (and) cetearyl alcohol | 1.50 g |
| (and) C12-15 pareth-7 (and) PPG-25 laureth-25 | |
| (Cosmacol PSE): | |
| Cyclohexasiloxane: | 10.00 g |
| Stearyl alcohol: | 1.00 g |

Phase B

| | |
|---|---|
| Preservatives: | 0.75 g |
| Pentasodium ethylene diamine tetramethylene | 0.05 g |
| phosphate: | |
| Ammonium polyacryldimethyltauramide | 0.40 g |
| (Hostacerine AMPS): | |
| Xanthan gum (Rhodicare S): | 0.20 g |
| Zinc gluconate (Labcatal) | 1.8 g |
| Water | qs |

### Procedure:

Heat phase B to approximately 75° C. and incorporate ammonium polyacryldimethyltauramide therein; stir until a homogeneous gel is obtained.
Heat phase A to approximately 75° C.
Produce the emulsion by incorporating phase A into phase B.

The composition is applied to the skin of the face and/or body and the area of skin to which said composition has been applied is massaged with a manual massage instrument of type Environ Cosmetic Roll-Cit. The duration of the massage may range from 1 minute to several minutes in order to optimize the required effect on the homeostasis of the skin and/or radiance of the complexion and/or the mechanical properties of the skin.

W/O/W triple emulsion
Primary emulsion (A):

| | | |
|---|---|---|
| Polyglyceryl-4 isostearate, hexyl laurate and | 3.50 | g |
| cetyl PEG/PPG 10/1 dimethicone: | | |
| Cyclopentasiloxane: | 16.50 | g |
| Dimethicone: | 4.00 | g |
| Magnesium sulphate | 0.80 | g |

| | |
|---|---|
| Water: | qs |

Multiple emulsion:

| | | |
|---|---|---|
| Primary emulsion (A): | 22.50 | g |
| Cyclopentasiloxane: | 3.50 | g |
| Apricot kernel oil: | 4.00 | g |
| Water: | 65.05 | g |
| Preservatives | 1.00 | g |
| Pentasodium ethylene diamine tetramethylene | 0.05 | g |
| phosphonate: | | |
| Alkyl acrylate copolymer: | 0.60 | g |
| Sodium hydroxide: | 0.30 | g |
| Peptide Gly-Pro-Gln-Gly-Pro-Gln (Collaxyl ®) | 3 | g |

### Procedure

### Preparation of the Primary Emulsion:

At ambient temperature and with stirring, the Polyglyceryl-4 isostearate, the hexyl laurate, the cetyl PEG/PPG 10/1 dimethicone, the cyclopentasiloxane and the dimethicone are homogenized. With vigorous stirring, the water and the ethylenic copolymer of the invention are incorporated slowly.

### Preparation of the Triple Emulsion:

At ambient temperature and with stirring, the alkyl acrylate copolymer, the preservatives and the sequestrant (pentasodium ethylene diamine tetramethylene phosphonate) are dispersed. The mixture is left to swell for approximately 45 minutes with stirring and then neutralized with sodium hydroxide. The primary emulsion is diluted with the cyclopentasiloxane and the apricot kernel oil, and then this mixture is incorporated slowly with stirring into the aqueous phase.

This composition is applied to the areas of the body where the skin is soft and/or flaccid, particularly the stomach and/or thighs. Subsequently a massage is carried out using a Lift6 device, for a period ranging from 10 to 30 minutes, in order to optimize the required effect on the elasticity and/or firmness of the skin.

Water-in-oil emulsion

| | | | |
|---|---|---|---|
| A- | Polymethylcetyl dimethyl methylsiloxane, | 1.5 | g |
| | oxyethylenated | | |
| | Isostearate, polyglycerolated | 0.5 | g |
| | Isohexadecane | 4 | g |
| | Squalane | 1.85 | g |
| | Dimethicone | 2.05 | g |
| | Apricot kernel oil | 1.1 | g |
| | Cyclopentasiloxane | 9 | g |
| | Propylparaben | 0.15 | g |
| B- | Water | 29.2 | g |
| | Propylene glycol | 3 | g |
| | Magnesium sulphate | 1.75 | g |
| | Methylparaben | 0.2 | g |
| | Preservative | 0.3 | g |
| | Peptide Lys-Leu-Asp-Ala-Pro-Thr (Vinci 02) | 1.5 | g |
| C- | Nylon 12 | 3 | g |

### Procedure:

Homogenize phase A and phase B separately with stirring at ambient temperature.
Produce the emulsion by incorporating phase B into phase A.
Incorporate phase C with stirring.

In the morning the area of skin to be treated is massaged with a Environ Cosmetic Roll-Cit manual massage instrument. The duration of the massage may range from 1 minute to several minutes, in order to optimize the required effect on the homeostasis of the skin and/or the radiance of the complexion and/or the mechanical properties of the skin. The composition described above is also applied in the evening.

1-27. (canceled)

28. A method for cosmetic skin care comprising
applying to or on the skin:
(i) a composition comprising at least one agent which increases the expression of mechanoreceptors in the cells of the skin, and
(ii) a device to apply and/or maintain a controlled mechanical stress on the skin, wherein
the applying of the composition and the device is simultaneous or sequential.

29. The method for cosmetic skin care according to claim 28, wherein the controlled mechanical stress is at least one selected from the group consisting of a tension, a traction and a pressure.

30. The method for cosmetic skin care according to claim 28, wherein the device induces at least one stimulation selected from the group consisting of a mechanical stimulation, an electrical stimulation and an ultrasonic stimulation.

31. The method according to claim 30, wherein
the device induces a mechanical stimulation, and
the device is a manual massage instrument or a massage instrument which requires provision of external energy.

32. The method according to claim 30, wherein
the device induces a mechanical stimulation, and
the device is an adhesive support which maintains tension on the skin.

33. The method according to claim 32, wherein the adhesive support has an elastic modulus ranging from 500 MPa to 10,000 MPa.

34. The method according to claim 33, wherein the adhesive support has an elastic modulus ranging from 500 MPa to 2,000 MPa.

35. The method according to claim 28, wherein the at least one agent which increases the expression of mechanoreceptors in the cells of the skin is an agent which increases the expression of integrins in the cells of the skin.

36. The method according to claim 35, wherein the agent which increases the expression of integrins in the cells of the skin is a peptide.

37. The method according to claim 36, wherein the peptide is one selected from the group consisting of a mimetic peptide of fibronectin, a mimetic peptide of collagen and a mimetic peptide of laminin.

38. The method according to claim 37, wherein the peptide is one selected from the group consisting of a fibronectin mimetic peptide of sequence (AA)ₙ-Leu-Asg-Ala-Pro-(AA)ₙ in which AA is any amino acid or derivative thereof and n is between 0 and 2; a collagen mimetic peptide of sequence (Gly-Pro-Gln)ₙ-NH₂ in which n is between 1 and 3; a laminin mimetic peptide of sequence X₁-Y-Phe-Thr-X₂-Ala-Thr-Z-Ile-X₃-Leu-X₄-Phe-Leu-X₅ in which X₁, X₂, X₃, X₄ and X₅=Arg, Lys or His, Y=Asp or Glu and Z=Asn or Gln and mixtures thereof.

39. The method according to claim 35, wherein the agent which increases the expression of integrins in the cells of the skin is one selected from the group consisting of a zinc salt, a copper salt, a manganese salt, a derivative thereof and a mixture thereof.

40. The method according to claim 28, wherein a relative percent by weight composition of the at least one agent which increases the expression of mechanoreceptors in the cells of the skin is from 0.01% to 20%.

41. A cosmetic kit comprising:
(i) a composition comprising at least one agent which increases the expression of mechanoreceptors in the cells of the skin, and
(ii) a device to apply and/or maintain a controlled mechanical stress on the skin.

42. The cosmetic kit according to claim 41, wherein a reservoir of the device contains the composition comprising at least one agent which increases the expression of mechanoreceptors in the cells of the skin.

43. The method for cosmetic skin according to claim 28, wherein an improvement to the skin by applying the device for a controlled mechanical stress is potentialized and/or prolonged by applying composition comprising at least one agent which increases the expression of mechanoreceptors in the cells of the skin, and the improvement is at least one selected from the group consisting of improvement in a thickness of the skin, improvement in a radiance of the complexion, improvement in a density of the skin, improvement in a firmness, elasticity and tonicity of the skin, improvement in a regeneration of the skin and improvement of cicatrisation of the skin.
